(19) United States
(12) Patent Application Publication
(10) Pub. No.: US 2001/0004709 A1
(43) Pub. Date: Jun. 21, 2001

(54) TISSUE EXPANDER APPARATUS WITH MAGNETICALLY LOCATABLE INJECTION SITE AND METHODS FOR USE

(76) Inventor: WILLIAM R. DUBRUL, REDWOOD CITY, CA (US)

Correspondence Address: WILLIAM R DUBRUL, P O BOX 246, REDWOOD CITY, CA, 94064

(21) Appl. No.: 09/326,412

(22) Filed: Jun. 4, 1999

Related U.S. Application Data
(60) Provisional application No. 60/088,558, filed on Jun. 9, 1998.

(51) Int. Cl. A61F 2/12

(52) U.S. Cl. 623/8; 623/23.72

(57) ABSTRACT

Devices with sites that are located using a magnetic field.

This patent claims priority of Provisional Pat. Application Ser. No. 60/088,558 filed Jun. 9, 1998.

### BACKGROUND OF THE INVENTION

1. Field of the Invention

The present idea relates to medical devices and methods. In particular, the present idea relates to an improved tissue expander and method for its use, where the tissue expander has an injection site that is implanted beneath the skin and said injection site is located externally from outside the patients body using magnetic field.

2. Description of the Background Art

The prior art is well aware of tissue expanders and the methods for using them with over fifty U.S. Pats. having been issued on the technique and the devices that are used. Some pertinent descriptions are set forth in a number of issued U.S. Pats., including U.S. Pat. Nos. 4,217,889, 5,425,760, 5,074,878 as well as tissue expander patents by the present inventor, U.S. Pat. Nos. 4,798,584 and 4,671,255.

### SUMMARY OF THE INVENTION

The present invention provides improved tissue expanders of the type having an injection site located 'integrally' on the shell/envelope of the expander or remotely located away from the expansion envelope that is implanted beneath the skin of the patient. Both injection site systems are located using a magnetic field for the subsequent percutaneous injections for expansion of the tissue expander, tissue and creation of the tissue flap. The improvement comprises configuring the injection site of the tissue expander so that it has a magnetically locatable component in it so that it is detectable from outside of the patient's body. In this way an externally applied magnet locator can be used to find the injection site without using the more common, but cumbersome technique of palpation through the patient's skin, muscle, other tissues, etc.

Tissue expanders are a common tool used by general, plastic and reconstructive surgeons. They allow the surgeon to create additional tissue for reconstructive or cosmetic surgery instead of the much more cumbersome procedure of distant transfer flaps. With distant transfer flaps, the surgeon usually harvests tissue from a distant area (often the thigh or buttock area is used) from where it is needed and transplants the tissue/graft to the locale on the body where tissue is needed. This often requires difficult microsurgery for reconnecting blood vessels so that the transferred tissue graft will survive. Additionally the tissue characteristics of the distant graft may not match those characteristics of the skin (and tissue) where the additional tissue is needed.

It is an object of the present invention to provide a tissue expander having a 'self-contained', 'integral' injection site, reservoir, valve etc. in which the injection sites are readily located after implantation.

It is yet another object of the present invention to provide a tissue expander with an integral injection site whereby the danger of injecting into the expansion envelope, balloon, shell, etc. is minimized.

It is yet another object of the present invention to provide a tissue expander with a remote injection site that is less palpable than conventional sites and location by the physician is accomplished using a magnetic field.

It is yet another object of the present invention to provide a tissue expander that has a minimal amount of hard, reinforced, etc. palpable material that may cause deleterious tissue erosion to the patient.

A tissue expander having a thin expandable envelope with the injection site attached to the envelope directly or via a connecting tube attains the foregoing and other objects and advantages.

When the injection site is directly attached to the envelope, as with an integrally locatable injection site, it may be preferred that the base of the envelope has a thicker dimension than that of the anterior (or lateral) surface of the expansion envelope. In this instance, the expansion envelope will have a tendency to remain somewhat flat and soft within the pocket created by the surgeon at the time of implantation. This tendency will minimize the potential for the thinner portion of the anterior surface of the expander envelope to 'fold over' onto the top of the expander. Preventing this 'fold over' is significant so that when the physician is incrementally filling the expander, he or she does not iatragenically puncture the expansion envelope of the tissue expander with the small gauged needle that is used for the periodic percutaneous fillings. Further, because the base of the expander is thicker or reinforced as opposed to the anterior surface, the anterior surface of the expander will remain soft against the expanding tissue thus avoiding erosion possibilities.

When the injection site or reservoir is 'remote', that is when it is attached to the expansion reservoir via a small connecting tube, it is preferred that the remote site not be too large so that it will not be too difficult to tunnel under the patients skin and muscle. Additionally and probably more important is that the remote site does not have a tendency to erode through the skin and/or muscle during the implantation/expansion period that approximates 90 days. The present invention provides for an injection site (remote or integral) that does not require 'palpation' for the physician to locate it under the skin. Instead a magnetic field is used. This allows the injection site to be very friendly to the overlying tissue or even invisible to it.

### BRIEF DESCRIPTION OF THE DRAWINGS

FIG. 1 is a schematic illustration of a tissue expander constructed in accordance with the principles of the present invention where an integral injection site is used.

FIG. 2 is an illustration of a tissue expander constructed in accordance with the present invention where an integral injection site is used. In this drawing, the expander is implanted under the patient's tissue and an external magnetic locator is being used to locate the injection site externally.

FIG. 3 is an illustration of the tissue expander of the present invention where the injection site is of the remote style. In this drawing, the external magnetic locator is being used to locate the remote injection site externally while the expander is implanted internally beneath the patient's tissue.

### DESCRIPTION OF THE EXEMPLARY EMBODIMENTS

The tissue expander of the present invention is used for creating tissue for reconstructive or cosmetic surgery. It is used beneath the tissue (skin, muscle, etc.) of a patient and usually below the muscle so that both the skin and muscle is stretched/expanded. It has also been shown that new tissue is actually created using the tissue expander. Usually the tissue expander is placed beneath the tissue in the operating room and after a week or so, the patient returns to the physician's office or hospital for subsequent inflations usually with sterile isotonic saline solution. These inflations usually occur in a sterile environment using a small gauge needle (˜25 G) that is placed percutaneously through the skin, past a resealing septum and into the tissue expander. The number, frequency and amount of inflations vary from patient to patient. Further, the location of where the tissue is being expanded on the patient's body has an important effect on these inflation rates and amounts as well. As a general rule, the tissue of younger patients and the elderly expand with more ease than do the tissue of patient's in the middle age range. Further, tissue located near parts of the body where vascularity is sound usually expand easier. For example the head, neck and chest areas tend to expand more easily than do the areas of the lower extremities and feet. Even further, these inflation rates and amounts vary greatly with the thickness and quality of the tissue over the tissue expander that is being expanded.

Usually, the patient visits the physicians office every ten days or so where 20-100 cc of saline is added to the tissue expander. Tissue expansion is usually completed in less than three months. At that time the patient usually returns to the operating room, where the tissue expander is removed and the expanded tissue is used for its intended purpose. The intended purpose varies. The expanded flap can be used for the repair of burned tissue, breast reconstruction after mastectomy, for tattoo removal, nose reconstruction, distant transfer grafts, etc. Basically anywhere that additional tissue is required for skin closure, a tissue expander can be used.

Another somewhat new technique for use with tissue expanders is their use 'Intra-operatively'. Intra-operative use of tissue expanders is a technique whereby tissue expansion occurs very rapidly in the operating room. This is usually effective for expanding only small areas of tissues for eliminating small tissue defects.

The present invention provides for a tissue expander with an injection site that is not palpable by the physician. Further, the non-palpable injection site will not erode through the tissue being expanded. Often, the tissue being expanded becomes taught immediately upon insufflation by the physician. This taught tissue could become thinned as well. With any palpable bulge, rings, etc., there is a tendency for this palpable 'bulge' to erode through the patient's skin, which often causes more trouble than the defect that originally existed and the reason the tissue expander was originally chosen. The use of a magnetically locatable injection site obviates the potential for that erosion. Further, often the palpable injection site of a conventional tissue expander cannot be palpated because of fluids, hematomas or merely significant tissue that lies over the injection site. This is an additional reason that an injection site with a magnetically locatable injection site is useful. A tissue expander of the present inventor, U.S. Pat. No. 4,671,255 describe a tissue expander with a reinforced anterior surface of the expansion envelope. In that patent the inventor describes a magnetically locatable injection site designed into the hard-reinforced insert. This invention has its application in some areas of tissue expansion, but has deleterious effects due to the design of the anterior reinforcing insert. The reinforcing anterior insert with a magnetically locating injection site is often difficult to insert into the patient at the time of implantation. The reinforced anterior insert is difficult to fold and place through a small incision. Hence a larger incision is required. When using tissue expansion, it is preferred to use as small of an incision as possible. The larger the incision used, the longer the physician has to wait before beginning inflation of the device. Further, the duration between insufflations is often increased as well due to the larger incision. Even further, larger incisions have a tendency to open up much more readily than do the smaller incisions due to the forces realized to the tissue during the expansion process.

Further, and of great import, the previously invented hard-reinforced insert with a magnetically located injection site has a harder insert on the anterior surface. This harder surface can cause the aforementioned deleterious erosion through the overlying tissue.

Further, for many reasons, the physician prefers a remote injection site or port. This reason for this preference is several-fold. Only one of these reasons is that tissue expanders are often used to reconstruct tissue that has been removed or necrosed because of cancer (e.g. skin cancer (squamous cell carcinoma), breast cancer (mastectomy), etc.). Often when tissue is damaged from carcinogenic therapies, harsh radiation therapy is an accompanying treatment. Once tissue has been subjected to this harsh radiation therapy, the tissue becomes extremely frail. It can be expanded; however, expansion must be done with great care. Due to the frailness of the tissue during expansion, repeated punctures of the frail tissue under tension due to the expansion with the percutaneous needle that is used for inflations must be avoided. Hence, a remotely located injection site is preferred. The advantage of a remote site that can be located without requiring palpability is desired.

For at least these reasons, an improved device that has no reinforced anterior surface is desired. Further, a remote injection site for tissue expanders with a magnetically locatable injection site is desired for reasons mentioned above as well.

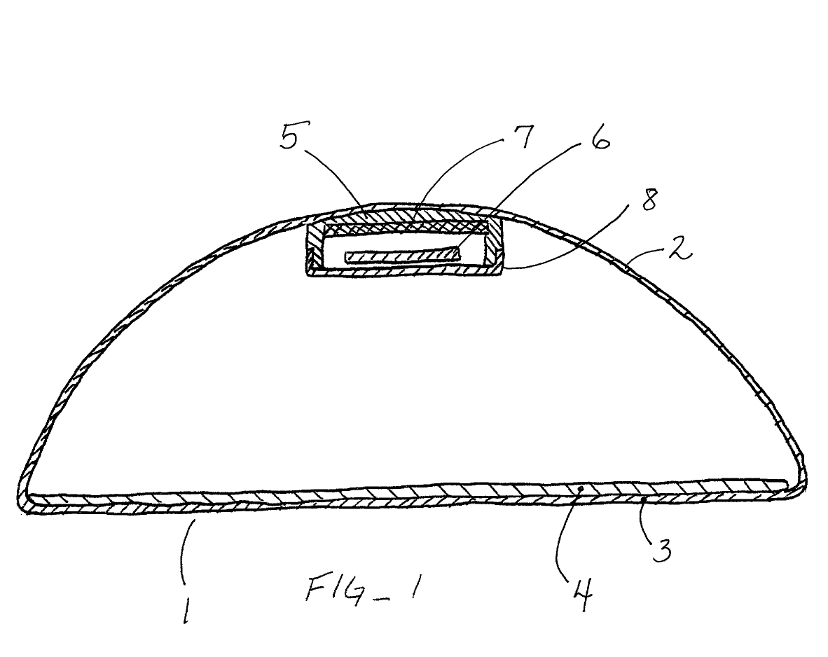

Turning now to FIG. 1, a schematic illustration of the instant invention is shown. The tissue expander 1 of the instant invention is illustrated. Most often, the tissue expander is comprised of a flexible envelope 9 that will be empty and with minimal volume when implanted into the patient. The expansion envelope is often constructed using medical grade, biocompatible silicone elastomer. Silicone elastomer has been used in the medical device industry for over three decades and is well proven. It has been used to manufacture tissue expanders for over two decades. One common manufacturing technique used to make the envelope is to dip or cast liquid silicone over a mandril that is shaped in the manner with which you would like the envelope to be shaped (i.e. ovaloid, spheroid, rectanguloid, etc.). The liquid is allowed to cure to a solid form usually from 0.005-0.020 inches thick and the envelope is peeled off the mandril. An injection site is communicated with the envelope and the entire system is sealed to allow filling (through the injection site) without any leakage. During the tissue expansion period (while implanted) usually about 6-12 weeks, the tissue expander is inflated with a sterile liquid (usually sterile isotonic saline). These injections occur through the tissue and the self sealing injection site 5 using a needle (usually 21 G or smaller) that is placed percutaneously through the tissue and into the injection site 5. The expander is filled until the physician is comfortable with the tension that is created in the overlying tissue. The patient is sent home only to return after 6-12 days for another injection of saline until enough tissue ahs been generated. The expander is usually removed and the new generated tissue is used for the subsequent operation. Sometimes the tissue expander is left in place in the case of breast reconstruction due to mastectomy due to breast cancer. The tissue expander 1 of the instant invention has an anterior surface 2 that is often called a shell or envelope and a posterior surface 3 often referred to as shell or envelope bottom. The instant invention will usually have a reinforced base 4 of the expander envelope to help prevent the flexible shell from folding over onto the anterior surface 2. Without this reinforced base the needle could pierce through the flexible shell/envelope 9 if it were to fold over the anterior surface and be oriented between the injection site 5 and the overlying tissue. The instant invention will also have a magnetically detectable component 6 in the injection site 5 so that the injection site 5 can be located via a magnetic field from outside the body. Usually this magnetically detectable component is composed of a strong magnet such as rare earth, samarium-cobalt or neodymium, but any magnetically detectable material could be used. The stronger the magnetically detectable material, the greater the distance where the injection site can be located beneath the tissue to be expanded. Usually, there is also an impenetrable needle guard 8 that helps prevent the percutaneously placed needle from penetrated the base of the injection site that would thusly penetrate the base of the expander envelope and thus damaging the expanding characteristics of the device requiring its removal and replacement. The anterior envelope 2 of the instant invention is smooth and soft to prevent any inadvertent erosion that may occur due to the expansion. This is an important embodiment of the instant invention. Previous tissue expanders with injections sites located within the shell/envelope of the tissue expander as in the tissue expander 1 are often referred to as integral injection sites; integral because the injection site is integral to the shell. To prevent 'fold-over' of the shell onto the injection site, others have used a hard anterior insert. This insert can have a deleterious effect on the patient. Once expansion has begun, the anterior tissue becomes taught. Any hard material can extrude through that taught tissue. Some manufacturers have solved this problem by having the physician actually suture the injection site to the overlying tissue during implantation and before closing the wound. These manufacturers have adhered 'suture tabs' to the anterior shell, usually around the injection site. This suturing is very difficult for the surgeon and often the surgeon would actually cut the suture tabs off of the anterior shell prior to implantation with the hopes that the shell would not 'fold-over' the top of the injection site. Hence, by providing a smooth anterior surface 2 of the instant invention, a superior and novel device is presented.

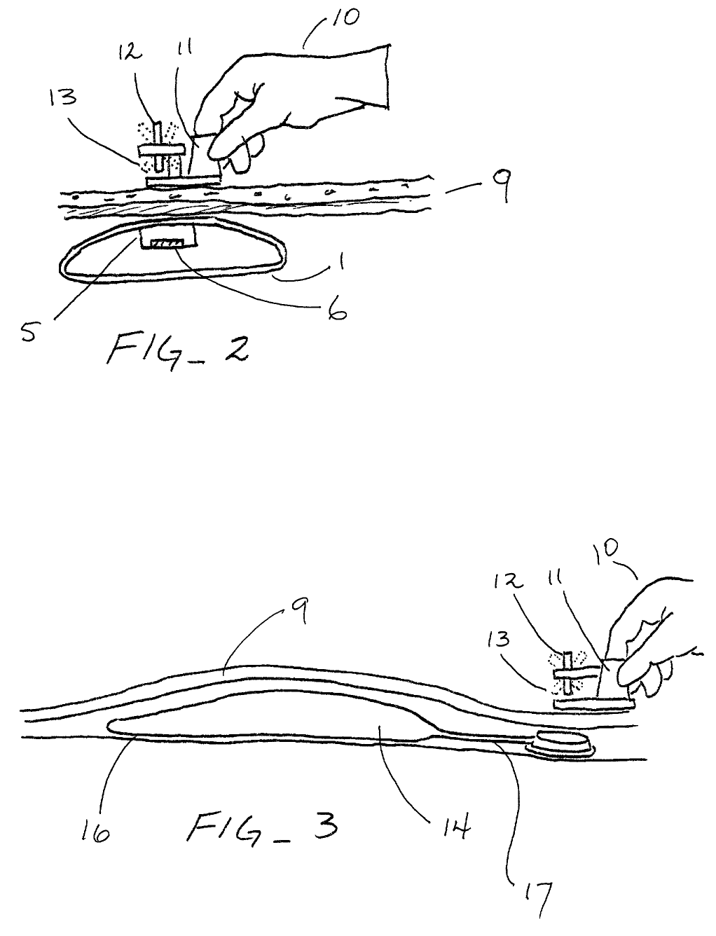

Turning now to FIG. 2, the tissue expander 1 of the instant invention is illustrated implanted in the body beneath the tissue 9. An external locator 11 is illustrated as being held in the hand 10 of the physician. In this case, the external magnetic locator 11 is composed of another magnetically detectable material 12 that is allowed to move freely with when placed in or near a magnetic field. This movement is illustrated by the dotted lines 13 in the drawing. This 'gimbaled' magnetic material is usually made of strong magnets as stated previously for the magnet in the injection site 5. The magnetic material 12 can be allowed to gimbal in one plane or two planes. When the locator gimbals in two planes, the center of the injection site is readily discernable when the locator is orthogonal to the tissue. When the locator is gimbaled in one plane, the injection site must be used twice; once to determine one line and then again perpendicular to the first line. When the gimbaled locator is orthogonal again then 'X' marks the spot.

Turning now to FIG. 3, a tissue expander 14 of the instant invention is illustrated. The tissue expander 14 illustrated in this figure is one where the injection site 15 is located away from the expansion envelope 16 usually connected by a tube 17. This is usually referred to as a 'remote tissue expander' because the injection site 15 is located 'remotely' from the expansion envelope 16. In FIG. 3, the tissue expander 14 is implanted in the body below the skin/tissue 9 to be expanded. The injection site 15 will likely have a needle guard (not illustrated) and a resealing septum (not illustrated) as well as a magnetically locatable material (also not illustrated). The injection site 15 is located in the same fashion as discussed above for FIG. 2. The physician's hand 10 holds the external magnetic locator 11 and passes it over the tissue until the gimbaled magnetic locator 12 moves in response to a magnetic field that emanates from the injection site 15. FIG. 3 illustrates a preferred embodiment of the present invention whereby the 'remote' injection site is located using a magnetic field locator 11 that is external to the body. In this instance, there exists no hard surface on the anterior surface of the tissue expander that could tend to cause problems with expansion of the tissue such as erosion during expansion; the predominant advantage of the present invention over pre-existing tissue expansion technologies.

An exemplary tissue expander has the following characteristics:

Working Length (ellipse, rectangular, cube, horseshoe-shaped etc.)

5-50 cm

Working Diameter (round, spherical, crescent, etc.)

10-30 cm

Working Volume when filled

50-2,000 cc

Physical Configuration

The tissue expander of the present invention can come in a variety of shapes. Common shapes are hemispheres, partial hemispheres, rectanguloids, crescents, ellipsoids, cubes, horseshoe-shaped, etc. However, a variety of shapes different from those mentioned above are common as well. The expansion envelope may be fabricated from several different materials some of which may be, but are not limited to silicone, latex, polyurethane, etc. These materials have elastomeric characteristics associated with them, but the envelope need not be made of only these. There are polyurethanes that have generally inelastic characteristics as well as a wide variety of other materials. It is important that the materials have a somewhat liquid proof characteristic to them however because of the periodic filling of fluids to create the expansion. Usually the wall thickness of the expansion envelope is small in the range of 0.003-0.015 inches thick.

Exemplary tissue expanders having magnetically locatable injection sites are illustrated in FIGS. 1, 2 & 3.

Although the foregoing invention has been described in some detail by way of illustration and example, for purposes of clarity of understanding, it will be obvious that certain changes and modifications may be practiced within the scope of the appended claims.

What is claimed is:

1. An improved device of the type having a site that is located using a magnetic field.

2. An improved device of claim 1, wherein the device is a medical device known as a tissue expander.

3. An improved device of claim 2, wherein the injection site is located on the expansion envelope of the tissue expander.

4. An improved tissue expander of claim 2, wherein the injection site is located away from the expansion envelope of the tissue expander.

5. An improved device of claim 3, wherein the injection site is located on the expansion envelope of the tissue expander and the base of the expander has increased stiffness.

6. An improved device of claim 1, wherein the device is a medical device for the percutaneous injection of fluids into the body.

7. An improved device of claim 6, wherein the site is a re-sealable septum that will receives a needle for percutaneous injections through the skin.

8. An improved device of claim 4, wherein the anterior surface of the tissue expander envelope is smooth and without reinforcement except for the injection site.

9. A method for locating a site in the body using a magnetic field that cannot otherwise be located via physical palpation.

10. A method of claim 9 where the site is an injection site of a tissue expander.

11. A method of claim 9, wherein said tissue expander has the injection site located on the anterior surface of the shell.

12. A method of claim 9, wherein the tissue expander injection site is located remotely from the shell.
